(19) United States
(12) Patent Application Publication
(10) Pub. No.: US 2008/0026053 A1
(43) Pub. Date: Jan. 31, 2008

(54) Capsule containing granular pharmaceutical compositions

(75) Inventors: Suresh Dixit, Fort Worth, TX (US); Ralph Brown, Southlake, TX (US); Juan Carlos Menendez, North Richland Hills, TX (US); Kripesh Munnirpallam, Fort Worth, TX (US)

Correspondence Address: GREENBLUM & BERNSTEIN, P.L.C., 1950 ROLAND CLARKE PLACE, RESTON, VA, 20191, US

(73) Assignee: Sovereign Pharmaceuticals, Ltd., Fort Worth, TX (US)

(21) Appl. No.: 11/494,616

(22) Filed: Jul. 28, 2006

(51) Int. Cl. A61K 9/52

(52) U.S. Cl. 424/457

(57) ABSTRACT

A capsule for oral administration to a human subject. The capsule comprises at least two different pharmaceutical compositions in granular form. This Abstract is neither intended to define the invention disclosed in this specification nor intended to limit the scope of the invention in any way.

### BACKGROUND OF THE INVENTION

1. Field of the Invention

The present invention relates to a capsule, in particular, a gelatin capsule, for oral administration to human subjects which contains at least two different granular pharmaceutical compositions and to a process for making the gelatin capsule.

2. Discussion of Background Information

Capsules are often preferred by patients over compressed tablets because they are easier to swallow. Immediately upon contact with the moisture in the human mouth they become exceedingly slippery and slide down the throat easily and without the friction on the mucus membrane associated with compressed tablets.

Capsules (usually gelatin capsules) that are currently available to prescribing physicians and dispensing pharmacists that incorporate in a single capsule two different pharmaceutical compositions such as, e.g., an immediate release composition and a sustained-release composition are made by very expensive methods of manufacture that require either fluid bed coaters or pharmaceutical manufacturing equipment that provides the compositions by extrusion and spheronization. These methods permit precise amounts of active pharmaceutical agents to be sprayed onto beads or to prepare pharmaceutical compositions in the form of pellets which are then put into the capsules.

In addition to the expensive equipment, these manufacturing methods require large batch sizes which—combined with the investment in capital goods required for their manufacture—limits the ability of small to medium size manufacturers to compete with larger and better capitalized companies.

Small pharmaceutical manufacturing firms, however, are usually well equipped for the manufacture of granular formulations which are traditionally compressed into tablets. Unlike beads or pellets that are used to fill variable release capsules that require expensive equipment for the manufacture thereof, the manufacturing equipment and processes used for making granular formulations are inexpensive and easily available.

One major (if not the only major) reason why granular formulations do not appear to have been used in capsules in the past is due to the fact that all granular formulations that require two or more different matrices such as, e.g., an immediate release matrix and a controlled release matrix, have been compressed into tablets. Because this is the traditional method of manufacturing pharmaceutical dosage forms using granular formulations, many of the inactive ingredients (excipients) commonly used in tablet formulations are too bulky—unless compressed by powerful tablet presses—to fit the required and/or desired quantity of the formulations into capsules of a size that is small enough to be suitable for oral ingestion by human subjects.

### SUMMARY OF THE INVENTION

According to the present invention it has been found that capsules which are suitable for oral ingestion by human subjects can be filled with two or more different active ingredient containing granular (granulated) compositions in an amount which is sufficient to provide a therapeutic effect by selecting at least as the major part of the inactive ingredients (excipients) of these compositions substances which exhibit a low bulkiness. This avoids the use of the expensive equipment and the large batch sizes that are required for the currently available bead or pellet containing capsules.

Accordingly, the present invention provides a capsule (preferably a gelatin capsule) for oral administration to a human subject. The capsule comprises at least two different pharmaceutical compositions in granular form.

In one aspect of the capsule, at least one of the at least two different compositions may be an immediate release composition and/or at least one of the at least two different compositions may be a controlled release composition.

In another aspect, the capsule may comprise at least two compositions which differ with respect to at least the release profiles thereof. For example, the at least two different pharmaceutical compositions may comprise the same active ingredient and provide two different release profiles of the active ingredient such as, e.g., an immediate release composition and a controlled release composition.

In yet another aspect of the capsule, the at least two different pharmaceutical compositions may comprise at least two different active ingredients. Further, one of the at least two compositions may be an immediate release composition and the other one may be a controlled release composition.

In another aspect of the capsule of the present invention, at least one or, preferably, each of the at least two compositions may comprise not more than about 40% by weight of one or more active ingredients, based on the total weight of a composition.

In another aspect, the capsule may comprise at least one immediate release composition comprising at least about 70% by weight, based on the total weight of all excipients of the composition, of one or more excipients selected from sugars and sugar alcohols, microcrystalline cellulose, dicalcium phosphate, starch and derivatives thereof, stearic acid and salts thereof and optionally crosslinked polyvinylpyrrolidone. In another aspect, the immediate release composition may comprise at least about 40% by weight of microcrystalline cellulose, based on the total weight of all excipients of the immediate release composition.

In yet another aspect, the capsule may comprise at least one controlled release composition comprising at least about 70% by weight of one or more excipients selected from microcrystalline cellulose, starch and derivatives thereof, cellulose ethers and/or esters, copolymers of esters of at least one of acrylic acid and methacrylic acid, sugars and sugar alcohols, alginates, gelatin, polyvinylalcohol, polyvinylpyrrolidone, and stearic acid and salts thereof. In another aspect, the controlled release composition may comprise at least about 40% by weight of microcrystalline cellulose, based on the total weight of all excipients of the controlled release composition.

In yet another aspect, the capsule of the present invention may comprise a total of not more than about 0.8 g, e.g., a total of not more than about 0.6 g, of the at least two different pharmaceutical compositions.

In a still further aspect of the capsule of the present invention, at least one of the at least two different pharmaceutical compositions may comprise one or more active ingredients selected from analgesics, antihistamines, decongestants, antitussives, expectorants, vitamins, anti-inflammatories, anti-infectives, antibiotics and laxatives.

In another aspect, at least one of the at least two different pharmaceutical compositions may have been obtained by a wet granulation method and/or at least one of the at least two different pharmaceutical compositions may have been obtained by a dry granulation method.

In another aspect, the capsule may be a soft gelatin capsule. In yet another aspect, the capsule may be a hard gelatin capsule.

In a still further aspect, the capsule may comprise a therapeutically effective amount of each active ingredient contained therein.

The present invention also provides a gelatin capsule for oral administration to a human subject which comprises at least two (and preferably only two) different pharmaceutical compositions which have been obtained by granulation of a mixture of one or more active ingredients and one or more excipients. At least one of the compositions is an immediate release composition and comprises at least one first active ingredient and at least one of the compositions is a controlled release composition and comprises at least one second active ingredient which is the same as or different from the first active ingredient. Further, the capsule comprises a therapeutically effective amount of the or each active ingredient contained therein.

In one aspect of this capsule, at least one or, preferably, each of the compositions may comprise not more than about 40% by weight of one or more active ingredients, based on the total weight of each composition.

In another aspect of the capsule, the immediate release composition may comprise at least about 70% by weight, based on the total weight of all excipients of the composition, of one or more excipients selected from sugars and sugar alcohols, microcrystalline cellulose, dicalcium phosphate, starch and derivatives thereof, stearic acid and salts thereof and optionally crosslinked polyvinylpyrrolidone. In another aspect, the immediate release composition may comprise at least about 40% by weight of microcrystalline cellulose, based on the total weight of all excipients of the immediate release composition.

In yet another aspect of the capsule, the controlled release composition may comprise at least about 70% by weight, based on the total weight of all excipients of the composition, of one or more excipients selected from microcrystalline cellulose, starch and derivatives thereof, cellulose ethers and/or esters, copolymers of esters of at least one of acrylic acid and methacrylic acid, sugars and sugar alcohols, alginates, gelatin, polyvinylalcohol, polyvinylpyrrolidone, and stearic acid and salts thereof. In another aspect, the controlled release composition may comprise at least about 40% by weight of microcrystalline cellulose, based on the total weight of all excipients of the controlled release composition.

In a still further aspect, the capsule may comprise a total of not more than about 0.6 g of the at least two pharmaceutical compositions.

In another aspect of the capsule, at least one of the at least two different pharmaceutical compositions may comprise one or more active ingredients selected from analgesics, antihistamines, decongestants, antitussives, expectorants, vitamins, anti-inflammatories, anti-infectives, antibiotics and laxatives.

The present invention also provides a process for making a capsule for administration to a human subject. The process comprises filling the capsule with at least two different pharmaceutical compositions, each of which has been obtained by a granulation method.

In one aspect of this process, at least one of the compositions may have been obtained by a wet granulation method and/or at least one of the compositions may have been obtained by a dry granulation method.

In another aspect, at least one of the compositions may comprise an immediate release composition and/or at least one of the compositions may comprise a controlled release composition.

In yet another aspect, the at least two different pharmaceutical compositions may comprise at least two different active ingredients, each of which may be comprised in only one composition or in more than one composition.

In another aspect of the process, at least one or, preferably, each of the compositions may comprise not more than about 40% by weight of one or more active ingredients, based on the total weight of a composition.

In another aspect of the process of the present invention, the capsule may be filled with at least one immediate release composition comprising at least about 70% by weight, based on the total weight of all excipients of the composition, of one or more excipients selected from sugar and sugar alcohols, microcrystalline cellulose, dicalcium phosphate, starch and derivatives thereof, stearic acid and salts thereof, polyvinylpyrrolidone and crosslinked polyvinylpyrrolidone. In another aspect, the immediate release composition may comprise at least about 40% by weight of microcrystalline cellulose, based on the total weight of all excipients of the immediate release composition.

In yet another aspect of the process, the capsule may be filled with at least one controlled release composition comprising at least about 70% by weight, based on the total weight of all excipients of the composition, of one or more excipients selected from microcrystalline cellulose, starch and derivatives thereof, cellulose ethers and/or esters, copolymers of esters of at least one of acrylic acid and methacrylic acid, sugars and sugar alcohols, alginates, gelatin, polyvinylalcohol, polyvinylpyrrolidone, and stearic acid and salts thereof. In another aspect, the controlled release composition may comprise at least about 40% by weight of microcrystalline cellulose, based on the total weight of all excipients of the controlled release composition.

In a still further aspect, the capsule may be filled with a total of not more than about 0.8 g of the at least two different pharmaceutical compositions.

### DETAILED DESCRIPTION OF THE PRESENT INVENTION

The (preferably gelatin) capsule for oral administration to a human subject which constitutes one aspect of the present invention comprises at least two (e.g., two, three or four and preferably only two) different pharmaceutical compositions in granular (granulated) form. At least one of the at least two different compositions may be an immediate release composition and/or at least one of the at least two different compositions may be a controlled release composition.

In one aspect, the capsule may comprise at least two compositions which differ with respect to at least the release profiles thereof. For example, the at least two different pharmaceutical compositions may comprise the same active ingredient and provide two different release profiles of the active ingredient such as, e.g., an immediate release composition and a controlled release composition or two different controlled release compositions.

The term “controlled release composition” as used herein and in the appended claims refers to any composition that is not an immediate release composition, i.e., does not release all of the active ingredients contained therein within a relatively short time (for example, within less than 1 hour, e.g., less than 0.5 hours, following ingestion of the capsule). Accordingly, this term is a generic term which encompasses, e.g., sustained (extended) release compositions, pulsed release compositions, delayed release compositions, and the like. Preferably, the controlled release compositions for use in the present invention release the one or more active ingredients contained therein continuously or intermittently and, preferably, in approximately equal amounts per time unit, over an extended period of time such as, e.g., at least about 2 hours, at least about 3 hours, at least about 4 hours, or at least about 6 hours. The desirable length of the time period of continuous or intermittent (e.g., pulsed) release depends, inter alia, on the plasma half-life of the active ingredient and/or an active metabolite thereof. By way of non-limiting example, a capsule of the present invention may comprise a pharmaceutical composition comprising an active ingredient about 70-90% of which are released upon contact of the composition with water over a period of from about 45 to about 240 minutes. A second composition may release the same or a different active ingredient in a controlled manner over a predetermined time. For example, the active ingredients may be formulated by using one or more swellable excipients which gel in the presence of water to achieve predetermined release characteristics.

In yet another aspect, the at least two different pharmaceutical compositions may comprise at least two different active ingredients. For example, at least one of the at least two different active ingredients may be present in only one of the compositions. Alternatively or cumulatively, at least one of the at least two different active ingredients may be present in at least two of the compositions. For example, one of the at least two compositions may be an immediate release composition and the other one may be a controlled release composition, or both of the at least two compositions may be two different controlled release compositions.

In another aspect of the capsule of the present invention, at least one or, preferably, each of the at least two compositions may comprise not more than about 40% by weight, of one or more (e.g., one, two, three or four) active ingredients, based on the total weight of a composition. For example, each of the compositions (or at least one composition) may comprise not more than about 35% by weight, not more than about 30% by weight, not more than about 25% by weight, or not more than about 20% by weight, of one or more active ingredients. The active ingredients may be present in various forms such as, where applicable, as free acid or salt thereof, as free base or salt thereof, or as ester.

In another aspect, the capsule may comprise at least one immediate release composition comprising at least about 70% by weight, e.g., at least about 75% by weight, at least about 80% by weight, at least about 85% by weight, or at least about 90% by weight, based on the total weight of all excipients of the composition, of one or more (e.g., one, two, three, four or five) excipients selected from sugars and sugar alcohols (such as, e.g., sucrose, lactose, dextrose, fructose, mannitol, sorbitol, xylitol and any combinations of two or more thereof), microcrystalline cellulose, dicalcium phosphate, starch and derivatives thereof, stearic acid and salts thereof (for example, alkaline earth metal salts such as, e.g., magnesium stearate) and optionally crosslinked polyvinylpyrrolidone. In another aspect, the immediate release composition may comprise at least about 40% by weight, e.g., at least about 45% by weight, at least about 50% by weight, at least about 55% by weight, or at least about 60% by weight, of microcrystalline cellulose, based on the total weight of all excipients of the immediate release composition.

In yet another aspect, the capsule may comprise at least one controlled release composition comprising at least about 70% by weight, e.g., at least about 75% by weight, at least about 80% by weight, at least about 85% by weight, or at least about 90% by weight, based on the total weight of all excipients of the composition, of one or more (e.g., one, two, three, four or five) excipients selected from microcrystalline cellulose, starch and derivatives thereof, cellulose ethers and/or esters (such as, e.g., methylcellulose, carboxymethylcellulose, hydroxypropylcellulose and hydroxypropylmethylcellulose, HPMC, also called hypromellose), copolymers of esters of at least one of acrylic acid and methacrylic acid (such as, e.g., one or more of the Eudragit grades), sugar and sugar alcohols (such as, e.g., sucrose, lactose, glucose, fructose, mannitol, sorbitol, xylitol and any combinations of two or more thereof), alginates, gelatin, polyvinylalcohol, polyvinylpyrrolidone, stearic acid and salts thereof (for example, alkaline earth metal salts such as, e.g., magnesium stearate). In another aspect, the controlled release composition may comprise at least about 40% by weight, e.g., at least about 45% by weight, at least about 50% by weight, at least about 55% by weight, or at least about 60% by weight, of microcrystalline cellulose, based on the total weight of all excipients of the controlled release composition.

In yet another aspect, the capsule of the present invention may comprise a total of not more than about 0.8 g, e.g., a total of not more than about 0.75 g, a total of not more than about 0.7 g, a total of not more than about 0.65 g, a total of not more than about 0.6 g, a total of not more than about 0.55 g, or a total of not more than about 0.5 g, of the at least two different pharmaceutical compositions.

In a still further aspect of the capsule of the present invention, at least one of the at least two different pharmaceutical compositions may comprise one or more (e.g., one, two, three, four or five) active ingredients selected from analgesics, antihistamines, decongestants, antitussives, expectorants, vitamins, anti-inflammatories (e.g., NSAIDs), anti-infectives, antibiotics and laxatives.

Non-limiting specific examples of active ingredients which are suitable for use in the present invention include acrivastine, astemizole, azatadine, azelastine, bromodiphenhydramine, brompheniramine, carbinoxamine, carebastine, cetirizine, chlophedianol, chlorcyclizine, clemastine, chlorothen, chlorpheniramine, cyclizine, cyproheptadine, descarboethoxyloratadine, desloratadine, dexbrompheniramine, dexchlorpheniramine, dimethindene, diphenhydramine, diphenylpyraline, doxylamine, ebastine, epanastine, efletirizine, fexofenadine, hydroxyzine, isothipendyl, ketotifen, loratadine, meclizine, methapyrilene, mizolastine, mequitazine, mianserin, montelukast, noberastine, norastemizole, picumast, phenindamine, pheniramine, phenyltoloxamine, promethazine, prophenpyridamine, pyrilamine, temelastine, terfenadine, thenyidiamine, thonzylamine, trimeprazine, tripelennamine, triprolidine, phenylephrine, pseudoephedrine, codeine, dihydrocodeine, hydrocodone, dextromethorphan, carbetapentane, chlophedianol, benzonatate, caramiphen, noscapine, guaifenesin, hyoscyamine, aspirin, methyl salicylate, diflunisal, benorylate, faislamine, amoxiprin, acetaminophen, diclofenac, indomethacin, sulindac, carprofen, fenoprofen, flurbiprofen, ibuprofen, ketoprofen, ketorolac, loxoprofen, naproxen, sodium naproxen, thiaprofenic acid, mefenamic acid, meclofenamic acid, phenylbutazone, oxyphenylbutazone, piroxicam, meloxicam, celecoxib, parecoxib, etoricoxib, nimesulide, ethenzamide, dexamethasone, prednisolone, betamethasone, hydrocortisone, fludrocortisone, bisacodyl, vitamins A, B, D, E and K, hydrocodone, oxycodone, morphine, meperidine, fentanyl, methscopolamine, amikacin, gentamicin, kanamycin, neomycin, netilmicin, streptomycin, trobamycin, loracarbef, ertapenem, imipenem, cilastatin, meropenem, cefadroxil, cefazolin, cephelaxin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicloplanin, vancomycin, azithromycin, clarithromycin, dirithromycin, erythromycin, troleandomycin, aztreonam, amoxicillin, ampicillin, aziocillin, carbenicillin, cloxacillin, dicloxacillin, flucloxacillin, meziocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethiozole, sulfasalazine, sulfisoxazole, trimethoprim, trimethoprim-sulfamethoxazole, demeclocycline, doxycycline, minocycline, oxytetracycline, tetracycline, chloramphnicol, clindamycin, ethambutol, fosfomycin, furazolidone, isoniazid, linezolid, metronidazole, nitrofurantoin, pyrazinamide, quinupristin, rifampin, spectinomycin, and pharmaceutically acceptable salts thereof. The term “pharmaceutically acceptable salt” as used herein refers to those salts of a particular drug that are not substantially toxic at the dosage administered to achieve the desired effect and do not independently possess significant pharmacological activity. Salts included within the scope of this term are, for example, pharmaceutically acceptable acid addition salts of a suitable inorganic or organic acid. Non-limiting examples of suitable inorganic acids are, for example hydrochloric, hydrobromic, sulfuric and phosphoric acids. Non-limiting examples of suitable organic acids include carboxylic acids, such as, e.g., acetic, propionic, tannic, glycolic, lactic, pyruvic, malonic, succinic, fumaric, malic, tartaric, citric, cyclamic, ascorbic, maleic, hydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acids, as well as sulfonic acids, such as methanesulfonic, ethanesulfonic, and β-hydroxyethanesulfonic acids.

In another aspect, at least one of the at least two different pharmaceutical compositions may have been obtained by a wet granulation method and/or at least one of the at least two different pharmaceutical compositions may have been obtained by a dry granulation method.

There are two main types of dry granulation methods. Either a large tablet (“slug”) is produced in a tablet press or the powder is squeezed between two rollers to produce a sheet of material (roller compaction). In both cases these intermediate products are broken using a suitable milling technique to produce granular material, which is usually sieved to separate the desired size fraction.

Wet granulation usually involves the massing of a mix of dry primary powder particles using a granulating fluid. The fluid contains a solvent which must be volatile so that it can be removed by drying, and must be non-toxic. Typical examples of solvents include one or more of water, ethanol and isopropanol. In the traditional wet granulation method the wet mass is forced through a sieve to produce wet granules which are then dried. A subsequent screening step breaks agglomerates of granules and removes the fine material.

In another aspect, the capsule may be a soft gelatin capsule. In yet another aspect, the capsule may be a hard gelatin capsule. The capsule may, for example, be a one-piece capsule or a two-piece capsule. Soft gelatin capsules are usually prepared from gelatin, glycerin and water, and can absorb several times their own weight in water. Other non-limiting materials for making capsules of the present invention include cellulose esters and/or ethers such as, e.g., hydroxypropylmethylcellulose (HPMC).

In a still further aspect of the capsule of the present invention, the capsule may comprise a therapeutically effective amount of each active ingredient contained therein and preferably is capable of providing a plasma concentration within the therapeutic range of each active ingredient contained therein. The term “therapeutic range” as used herein refers to the range of active ingredient levels (including active metabolite levels) within which most patients will experience a significant therapeutic effect (including alleviation of symptoms) without an undesirable degree of adverse reactions.

The present invention also provides a gelatin capsule for oral administration to a human subject which comprises at least two (and preferably only two) different pharmaceutical compositions which have been obtained by granulation of a mixture of one or more (e.g., one, two, three, four or five) active ingredients and one or more (e.g., one, two, three, four, five or six) excipients. At least one of the compositions is an immediate release composition and comprises at least one first active ingredient and at least one of the compositions is a controlled release composition and comprises at least one second active ingredient which is the same as or different from the first active ingredient. Further, the capsule comprises a therapeutically effective amount of the or each active ingredient contained therein.

In one aspect of this capsule, at least one or, preferably, each of the compositions may comprise not more than about 40% by weight of one or more active ingredients, based on the total weight of each composition. For example, each composition (or at least one composition) may comprise not more than about 35% by weight, not more than about 30% by weight, not more than about 25% by weight, or not more than about 20% by weight, of one or more active ingredients.

In another aspect of the capsule, the immediate release composition may comprise at least about 70% by weight, e.g., at least about 75% by weight, at least about 80% by weight, at least about 85% by weight, or at least about 90% by weight, based on the total weight of all excipients of the composition, of one or more (e.g., one, two, three, four or five) excipients selected from sugars and sugar alcohols, microcrystalline cellulose, dicalcium phosphate, starch and derivatives thereof, stearic acid and salts thereof and optionally crosslinked polyvinylpyrrolidone. In another aspect, the immediate release composition may comprise at least about 40% by weight, e.g., at least about 45% by weight, at least about 50% by weight, at least about 55% by weight, or at least about 60% by weight, of microcrystalline cellulose, based on the total weight of all excipients of the immediate release composition.

In yet another aspect of the capsule, the controlled release composition may comprise at least about 70% by weight, e.g., at least about 75% by weight, at least about 80% by weight, at least about 85% by weight, or at least about 90% by weight, based on the total weight of all excipients of the composition, of one or more excipients selected from microcrystalline cellulose, starch and derivatives thereof, cellulose ethers and/or esters, copolymers of esters of at least one of acrylic acid and methacrylic acid, sugars and sugar alcohols, alginates, gelatin, polyvinylalcohol, polyvinylpyrrolidone, and stearic acid and slats thereof. In another aspect, the controlled release composition may comprise at least about 40% by weight, e.g., at least about 45% by weight, at least about 50% by weight, at least about 55% by weight, or at least about 60% by weight, of microcrystalline cellulose, based on the total weight of all excipients of the controlled release composition.

In a still further aspect, the capsule may comprise a total of not more than about 0.6 g, e.g., a total of not more than about 0.55 g, or a total of not more than about 0.5 g, of the at least two pharmaceutical compositions.

In another aspect of the capsule, at least one of the at least two different pharmaceutical compositions may comprise one or more active ingredients selected from analgesics, antihistamines, decongestants, antitussives, expectorants, vitamins, anti-inflammatories, anti-infectives, antibiotics and laxatives.

The present invention also provides a process for making a (preferably gelatin) capsule for administration to a human subject. The process comprises filling the capsule with at least two different pharmaceutical compositions, each of which has been obtained by a granulation method.

In one aspect of this process, at least one of the compositions may have been obtained by a wet granulation method and/or at least one of the compositions may have been obtained by a dry granulation method.

In another aspect, at least one of the compositions may comprise an immediate release composition and/or at least one of the compositions may comprise a controlled release composition.

In yet another aspect, the at least two different pharmaceutical compositions may comprise at least two different active ingredients, each of which may be comprised in only one composition or in more than one (such as, e.g., all) of compositions. By way of non-limiting example, one of the compositions may comprise a first active ingredient and another one of the compositions may comprise a second active ingredient, or one of the compositions may comprise a first active ingredient and a second active ingredient and another one of the compositions may comprise only the first active ingredient.

In another aspect of the process, at least one or, preferably, each of the compositions may comprise not more than about 40% by weight of one or more active ingredients, based on the total weight of a composition. For example, each composition (or at least one of the compositions) may comprise not more than about 35% by weight, not more than about 30% by weight, not more than about 25% by weight, or not more than about 20% by weight, of one or more active ingredients.

In another aspect of the process of the present invention, the capsule may be filled with at least one immediate release composition comprising at least about 70% by weight, e.g., at least about 75% by weight, at least about 80% by weight, at least about 85% by weight, or at least about 90% by weight, based on the total weight of all excipients of the composition, of one or more (e.g., one, two, three, four or five) excipients selected from sugars and sugar alcohols, microcrystalline cellulose, dicalcium phosphate, starch, stearic acid and salts thereof, polyvinylpyrrolidone and crosslinked polyvinylpyrrolidone. In another aspect, the immediate release composition may comprise at least about 40% by weight, e.g., at least about 45% by weight, at least about 50% by weight, at least about 55% by weight, or at least about 60% by weight, of microcrystalline cellulose, based on the total weight of all excipients of the immediate release composition.

In yet another aspect of the process, the capsule may be filled with at least one controlled release composition comprising at least about 70% by weight, e.g., at least about 75% by weight, at least about 80% by weight, at least about 85% by weight, or at least about 90% by weight, based on the total weight of all excipients of the composition, of one or more (e.g., one, two, three, four or five) excipients selected from microcrystalline cellulose, starch and eerivatives thereof, cellulose ethers and/or esters, copolymers of esters of at least one of acrylic acid and methacrylic acid, powdered sugar, alginates, gelatin, polyvinylalcohol, polyvinylpyrrolidone, and stearic acid and salts thereof. For example, the controlled release composition may comprise at least about 40% by weight, e.g., at least about 45% by weight, at least about 50% by weight, at least about 55% by weight, or at least about 60% by weight, of microcrystalline cellulose, based on the total weight of all excipients of the controlled release composition.

In a still further aspect, the capsule may be filled with a total of not more than about 0.8 g, e.g., a total of not more than about 0.75 g, a total of not more than about 0.7 g, a total of not more than about 0.65 g, a total of not more than about 0.6 g, a total of not more than about 0.55 g, or a total of not more than about 0.5 g, of the at least two different pharmaceutical compositions.

The granular compositions for use in the capsule of the present invention can be manufactured by wet and dry granulation processes which are well known to those of skill in the art. For example, the one or more active ingredients may be dispersed uniformly into a mixture of excipients, for example, by high shear granulation, low shear granulation, or fluid bed granulation. Excipients may include diluents, binders, disintegrants, dispersants, lubricants, glidants, stabilizers, surfactants and colorants. Non-limiting examples of diluents, also termed “fillers”, include lactose, cellulose, microcrystalline cellulose, mannitol, sorbitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Non-limiting examples of suitable binders include starch (including corn starch and pregelatinized starch), gelatin, sugars and sugar alcohols (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Non-limiting examples of lubricants include magnesium stearate, calcium stearate, stearic acid, talc, glyceryl behenate, sodium acetate, sodium lauryl sulfate, and polyethylene glycol. Non-limiting examples of disintegrants include starches, microcrystalline cellulose alginic acid, crosslinked polymers such as, e.g., crosslinked polyvinylpyrrolidone (crospovidone), crosslinked carboxymethylcellulose (croscarmellose) sodium, potassium or sodium starch glycolate, clays, celluloses, gums and the like. Non-limiting examples of suitable glidants include silicon dioxide, talc and the like. Stabilizers inhibit or retard drug decomposition reactions, including oxidative reactions. Surfactants may be anionic, cationic, amphoteric or nonionic. If desired, the pharmaceutical compositions for use in the present invention may also contain minor amounts of nontoxic auxiliary substances such as pH buffering agents, preservatives, e.g., antioxidants, wetting or emulsifying agents, solubilizing agents, coating agents, flavoring agents, and the like.

Extended/sustained release compositions may be made by choosing the right combination of excipients that slow the release of the active ingredients by coating or temporarily bonding or decreasing the solubility of the active ingredients. Examples of these excipients include cellulose ethers such as hydroxypropylmethylcellulose (e.g., Methocel K4M), polyvinylacetate-based excipients such as, e.g., Kollidon SR, and polymers and copolymers based on methacrylates and methacrylic acid such as, e.g., Eudragit NE 30D. According to the present invention care has to be taken that the bulkiness of the employed excipients as a whole is low enough to make it possible to incorporate at least two different granular pharmaceutical compositions (each of which comprising at least one active ingredient) into a capsule of a sufficiently small size for oral ingestion by a human subject. Preferably, the at least two compositions contained in a single capsule comprise a therapeutically effective amount of at least one, and preferably all, of the one or more active ingredients included therein. Of course, this does not exclude the possibility of including (preferably small amounts of bulky excipients into the compositions, as long as the overall bulkiness of the granular compositions is still low enough to permit incorporation of at least two different compositions in a single capsule that can be orally ingested by a human subject.

### EXAMPLES

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those skilled in the art how the several forms of the present invention may be embodied in practice.

### Example 1

A capsule in accordance with the present invention which comprises (a) 25 mg Promethazine HCl-IR (Immediate Release) and (b) 20 mg Phenylephrine HCl and 8 mg Chlorpheniramine Maleate-SR (Sustained Release) is illustrated as follows:

| | | |
|---|---|---|
| | | (AMOUNTS |
| | | FOR 1 Kg) |
| INGREDIENTS | Dose(mg) | in g |

SR GRANULES

| | | |
|---|---|---|
| PHENYLEPHRINE HCl | 20.000 | 111.12 |
| CHLORPHENIRAMINE MALEATE | 6.000 | 33.34 |
| MICROCRYSTALLINE CELLULOSE NF 50 | 48.000 | 266.69 |
| PURIFIED WATER | 12.000 | 66.67 |
| CHLORPHENIRAMINE MALEATE | 2.000 | 11.11 |
| POVIDONE K-30 USP | 3.500 | 19.45 |
| PURIFIED WATER | 1.000 | 5.56 |
| METHOCEL K4M PREM USP | 10.000 | 55.56 |
| METHOCEL K4M PREM USP | 9.000 | 50.00 |
| POLYETHYLENE GLYCOL | 1.500 | 8.33 |
| Totals | 100.000 | 555.6 |

IR-GRANULES

| | | |
|---|---|---|
| PROMETHAZINE HCL | 25.000 | 138.90 |
| PROSOLV SMCC 90 | 41.000 | 227.80 |
| PURIFIED WATER | 9.000 | 50.00 |
| POVIDONE K-30 USP | 3.000 | 16.67 |
| PROSOLV SMCC 90 | 9.500 | 52.70 |
| MAGNESIUM STEARATE NF | 1.500 | 8.33 |
| Totals | 80.000 | 444.4 |
| Totals | 180.000 | 1000.0 |

Procedure

### SR Granules

1. Prepare a solution using the scale up amounts of Purified Water, Chlorpheniramine Maleate and Povidone K-30.
2. Blend the dry mix scale up amounts of Phenylephrine HCl, Chlorpheniramine Maleate and Microcrystalline Cellulose NF 50 with a high shear mixer/granulator for 10 minutes (main propeller on, chopper off).
3. With the mixer/granulator on, pump the granulating solution prepared in step 1 into the mixer/granulator (main propeller on, chopper off). After completion, stop the mixer/granulator and rinse the container with Purified Water. Pump the rinse water to the mixer/granulator with main propeller and chopper on. Allow mixture to mix for an additional minute then turn mixer/granulator off.
4. Charge the post mix scale up amount of Methocel K4M Premium to the mixer/granulator and mix for 1 minute (main propeller and chopper on).
5. Dry the granulation overnight at 45° C. until the LOD (Loss On Drying) is 2% or less.
6. Resize the dried granulation through Fitzmill high speed # 93 screen knives forward.
7. Load the granules, Methocel and Polyethylene Glycol into a 20 ft³ V-blender.
8. Blend for 10 minutes.

### IR Granules

1. Prepare a solution using the scale up amounts of Purified Water and Povidone K-30.
2. Blend the dry mix scale up amounts of Promethazine HCl and Prosolv SMCC 90 with a high shear mixer/granulator for 10 minutes.
3. With the mixer/granulator on, pump the granulating solution prepared in step 1 into the mixer/granulator (main propeller on and chopper off).
4. After all solution has been added mix for an additional minute (main propeller and chopper on).
5. Dry the granulation overnight at 40° C. until the LOD is 2% or less.
6. Resize the dried granulation through Fitzmill high speed # 12 screen knives forward.
7. Load the granules and Prosolv SMCC 90 (a silicified microcrystalline cellulose obtainable from JRS Pharma LP) into a 20 ft³ V-blender and blend for 10 minutes.

### Final Blend

1. Load into the 20 ft³ V-blender: IR granules, SR granules.
2. Blend for 10 minutes.
3. Pass through oscillating granulator screen # 30 the Magnesium Stearate.
4. Load into the 20 ft³ V-blender the Magnesium Stearate and blend for 3 minutes.

### Final Subdivision

1. Fill into capsules size # 2.

### Example 2

A capsule in accordance with the present invention which contains (a) 0.275 mg Hyoscyamine Sulfate-SR and (b) 0.125 mg Hyoscyamine Sulfate-IR is illustrated as follows:

| | | |
|---|---|---|
| | | (AMOUNTS FOR |
| Ingredients | Dose(mg) | 1 Kg batch) in g |

SR Formula

| | | |
|---|---|---|
| LACTOSE MONOHYDRATE # 310 | 15.000 | 66.75 |
| PROSOLV SMCC 90 | 94.000 | 418.30 |
| PURIFIED WATER | 18.000 | 80.10 |
| HYOSCYAMINE SULFATE USP | 0.275 | 1.22 |
| POVIDONE K-30 USP | 6.000 | 26.70 |
| PURIFIED WATER | 2.000 | 8.90 |
| METHOCEL K4M PREM USP | 59.725 | 265.78 |
| Totals | 175.000 | 777.53 |

IR Formula

| | | |
|---|---|---|
| SODIUM STARCH GLYCOLATE NF | 2.000 | 8.90 |
| MICROCRYSTALLINE | 42.000 | 186.90 |
| CELLULOSE NF50 | | |
| PURIFIED WATER | 9.000 | 40.05 |
| HYOSCYAMINE SULFATE USP | 0.125 | 0.56 |
| POVIDONE K-30 USP | 2.875 | 12.79 |
| PURIFIED WATER | 1.000 | 4.45 |
| MAGNESIUM STEARATE NF | 3.000 | 13.35 |
| Totals | 50.000 | 222.50 |
| TOTAL FOR TWO GRANULATIONS | 225.000 | 1000.0 |

Procedure

### SR Granules

1. Prepare a solution using Purified Water, Hyoscyamine Sulfate and Povidone K-30.
2. Blend the dry mix scale up amounts of Lactose Monohydrate # 316 and Prosolv SMCC 90 with a high shear mixer/granulator for 10 minutes.
3. With the mixer/granulator on, pump the solution prepared in step 1 in to the mixer/granulator. After completion, stop the mixer/granulator and rinse the container with Purified Water. Pump the rinse water to the mixer/granulator with mixer on. Turn off mixer when completed.
4. Charge the post mix scale up amount of Methocel K4M Premium to the mixer/granulator and mix for 1 minute with chopper on.
5. Dry the granulation overnight at 45 C until the LOD is 2% or less.
6. Resize the dried granulation through Fitzmill high speed # 109 knives forward.

### IR-Granules

1. Prepare a solution using Purified Water, Hyoscyamine Sulfate and Povidone K-30.
2. Blend the dry mix scale up amounts of Sodium Starch Glycolate and Microcrystalline Cellulose NF50 with a high shear mixer/granulator for 10 minutes.
3. With the mixer/granulator on, pump the solution prepared in step 1 in to the mixer/granulator. After completion, stop the mixer/granulator and rinse the container with Purified Water. Pump the rinse water to the mixer/granulator with mixer on. Allow mixture to mix for an additional minute while keeping the chopper on.
4. Turn the mixer/granulator off. Resize the granules before placing into the tray oven by passing through Fitzmill fast speed screen 187 hammers forward.
5. Dry the granulation overnight until the LOD is 2% or less.
6. Resize the dried granulation through Fitzmill high speed # 109 knives forward.

### Final Blend

1. Load into the V-blender the IR granules and the SR granules.
2. Blend for 10 minutes.
3. Pass through oscillating granulator screen # 30 the Magnesium Stearate.
4. Load into the V-blender and blend for 3 minutes.

### Final Subdivision

1. Fill into capsules size # 1.

### Example 3

A capsule in accordance with the present invention which contains a total of 75 mg Phenylephrine HCl, 8 mg Carbinoxamine Maleate, and 30 mg Carbetapentane Citrate incorporated in two different sustained release matrices is illustrated as follows:

| | | |
|---|---|---|
| | | (AMOUNTS |
| | | FOR 1 Kg |
| INGREDIENTS | Dose(mg) | BATCH) IN g |

SR GRANULES-I

| | | |
|---|---|---|
| PHENYLEPHRINE HCl | 30.000 | 107.14 |
| CARBETAPENTANE CITRATE | 15.000 | 53.57 |
| CARBINOXAMINE MALEATE | 4.000 | 14.29 |
| METHOCEL K4M PREM USP | 16.000 | 57.14 |
| MICROCRYSTALLINE CELLULOSE | 40.000 | 142.86 |
| NF 50 | | |
| PURIFIED WATER | 14.000 | 50.00 |
| CARBINOXAMINE MALEATE | 4.000 | 14.29 |
| POVIDONE K-30 USP | 4.000 | 14.29 |
| PURIFIED WATER* | 1.000 | 3.57 |
| EUDRAGIT NE 30 D** | 15.000 | 53.57 |
| METHOCEL K4M PREM USP | 12.500 | 44.64 |
| Totals | 130.000 | 464.28 |

SR GRANULES-II

| | | |
|---|---|---|
| PHENYLEPHRINE HCl | 45.000 | 160.71 |
| CARBETAPENTANE CITRATE | 15.000 | 53.57 |
| MICROCRYSTALLINE CELLULOSE | 60.000 | 214.28 |
| NF50 | | |
| AQUACOAT ECD ETHYLCELLULOSE | 30.000 | 107.14 |
| USP** | | |
| METHOCEL | 18.000 | 64.29 |
| POLYETHYLENE GLYCOL 8000 | 3.000 | 10.71 |
| Totals | 150.000 | 535.71 |
| Totals | 280.000 | 1000.0 |

Procedure

### SR Granules I

1. Prepare a solution using the scale up amounts of Purified Water, Carbinoxamine Maleate and Povidone K-30.
2. Pour into a separate container the Eudragit NE 30 D, keep under moderate stirring while not in use.
3. Blend the dry mix scale up amounts of Phenylephrine HCl, Carbetapentane Citrate, Carbinoxamine Maleate, Methocel K4M and Microcrystalline Cellulose NF 50 with a high shear mixer/lodige mixer for 10 minutes (main propeller on, chopper off).
4. Pump the granulating solution prepared in step 1 into the high shear mixer/lodige mixer (main propeller on, chopper off). After completion, stop the mixer/granulator and rinse the container with purified water. Pump the rinse water into the high shear mixer/granulator with main propeller and chopper on.
5. Pump the Eudragit NE 30 D into the high shear mixer/granulator after pumping keep mixer on for additional minute (keep main propeller and chopper on).
6. Turn the equipment off and add the Methocel K4M Premium to the high shear mixer/lodige mixer and mix for 1 minute (main propeller and chopper on).
7. Dry the granulation overnight at 45° C. until the LOD is 2% or less.
8. Resize the dried granulation through Fitzmill high speed # 93 screen knives forward.

### SR Granules II

1. Pour the Aquacoat ECD into a separate vessel. Keep under continuous stirring to avoid settling down of solids.
2. Blend the dry mix scale up amounts of Phenylephrine HCl, Carbetapentane Citrate and Microcrystalline Cellulose NF50 with a high shear mixer/lodige mixer for 10 minutes (main propeller on and chopper off).
3. Pump the granulating solution (Aquacoat ECD) into the high shear mixer/lodige mixer (main propeller on and chopper off).
4. After all solution has been added mix for an additional minute (main propeller and chopper on).
5. Turn the equipment off and add the Methocel K4M Premium to the mixer/lodige mixer and mix for 1 minute (main propeller and chopper on).
6. Dry the granulation overnight at 45° C. until the LOD is 2% or less.
7. Resize the dried granulation through Fitzmill high speed # 93 screen knives forward.

### Final Blend

1. Load into the 20 ft³ V-blender: SR Granules I, SR Granules II.
2. Blend for 10 minutes.
3. Pass through oscillating granulator screen # 30 the Polyethylene Glycol 8000.
4. Load into the 20 ft³ V-blender the screened polyethylene glycol and blend for 5 minutes.

### Final Subdivision

1. Fill into capsules size # 1.

### Example 4

A capsule in accordance with the present invention which contains Aspirin in a delayed-release matrix and Aspirin and Caffeine in an immediate release matrix is illustrated as follows:

| | | |
|---|---|---|
| | | (AMOUNTS |
| | | IN 1 Kg- |
| Ingredients | Dose(mg) | batch) in g |

SR Formula

| | | |
|---|---|---|
| ASPIRIN | 81.000 | 188.37 |
| TALC | 0.750 | 1.74 |
| MICROCRYSTALLINE CELLULOSE NF90 | 10.249 | 23.83 |
| EUDRAGIT L 30 D-55 | 26.670 | 62.02 |
| Totals | 100.000 | 232.55 |

IR Formula

| | | |
|---|---|---|
| CAFFEINE ANHYDROUS | 20.000 | 46.51 |
| MICROCRYSTALLINE CELLULOSE NF90 | 24.000 | 55.81 |
| CROSPOVIDONE POLYPLASDONE | 15.000 | 34.88 |
| ASPIRIN | 245.000 | 569.75 |
| PURIFIED WATER | 55.000 | 127.90 |
| POVIDONE K-30 USP | 17.000 | 39.53 |
| STEARIC ACID | 9.000 | 20.93 |
| Totals | 330.000 | 767.42 |
| TOTAL FOR TWO GRANULATIONS | 430.000 | 1000.0 |

Procedure

### DR Granules

1. Keep Eudragit L 30-D 55 stirring before pumping into the dry mix.
2. Blend the dry mix scale up amounts of Caffeine and Aspirin, Talc and Microcrystalline Cellulose with a high shear mixer/lodige mixer for 10 minutes (main propeller on, chopper off).
3. With the mixer/granulator on, pump the Eudragit L 30 D-55.
4. After all the suspension has been pumped mix for an additional minute (main propeller and chopper on).
5. Dry the granulation overnight at 45° C. until the LOD is 2% or less.
6. Resize the dried granulation through Fitzmill high speed # 93 knives forward.

### IR Granules

1. Prepare a solution using Purified Water and Povidone K-30.
2. Blend the dry mix scale up amounts of anhydrous Caffeine and Microcrystalline Cellulose NF90 with a high shear mixer/granulator for 5 minutes (main propeller and chopper on).
3. Turn the high shear mixer/lodige mixer off. Load the following components into the high shear mixer/lodige mixer and blend for 10 additional minutes (main propeller on and chopper off).
4. Pump the granulating solution into the high shear mixer/lodige mixer. After completion keep the mixer/lodige mixer mixing for an additional minute (main propeller and chopper on).
5. Dry the granulation overnight until the LOD is 2% or less.
6. Resize the dried granulation through Fitzmill high speed # 93 knives forward.
7. Load into the V-blender (5-ft³ for high shear granules and 20-ft³): dry granules, scale-up amounts of Sodium Starch Glycolate and Microcrystalline Cellulose NF90.
8. Blend for 10 minutes.

### Final Blend

1. Load into the V-blender the IR Granules and the DR Granules.
2. Blend for 10 minutes.
3. Pass through oscillating granulator screen # 30 the Stearic Acid.
4. Load into the V-blender and blend for 5 minutes.

### Final Subdivision

1. Fill into capsules size # 0.

### Example 5

A capsule in accordance with the present invention which comprises (a) 10 mg Codeine Phoshate-IR and (b) 40 mg Codeine Phosphate, 50 mg Phenylephrine HCl, 8 mg Chlorpheniramine Maleate-SR is illustrated as follows:

| | | |
|---|---|---|
| | | (AMOUNTS |
| | | FOR 1 Kg |
| | | BATCH) |
| INGREDIENTS | Dose(mg) | IN g |

IR GRANULES

| | | |
|---|---|---|
| CODEINE PHOSPHATE | 10.000 | 39.22 |
| STARCH 1500 | 13.500 | 52.94 |
| MICROCRYSTALLINE CELLULOSE NF 50 | 54.000 | 211.76 |
| POVIDONE K-30 USP | 2.500 | 9.80 |
| PURIFIED WATER | 7.000 | 27.45 |
| Totals | 80.000 | 313.726 |

SR GRANULES

| | | |
|---|---|---|
| PHENYLEPHRINE HCl | 50.000 | 196.08 |
| CHLORPHENIRAMINE MALEATE | 8.000 | 31.37 |
| CODEINE PHOSPHATE | 40.000 | 156.86 |
| PROSOLV SMCC 90 | 34.500 | 135.29 |
| EUDRAGIT RS 30D | 66.667 | 261.44 |
| METHOCEL K4M PREMIUM | 20.000 | 78.43 |
| MAGNESIUM STEARATE | 2.500 | 9.80 |
| Totals | 175.000 | 686.28 |
| Totals | 255.000 | 1000.0 |

Procedure

### IR Granules

1. Prepare a solution using the scale up amounts of Purified Water and Povidone K-30.
2. Blend the dry mix scale up amounts of Codeine Phosphate, Starch 1500 and Microcrystalline Cellulose NF 50 with a high shear mixer/lodige mixer for 10 minutes (main propeller on, chopper off).
3. Pump the granulating solution prepared in step 1 into the high shear mixer/lodige mixer (main propeller on, chopper off). After completion and mix for additional minute (main propeller and chopper on).
4. Dry the granulation overnight at 45° C. until the LOD is 2% or less.
5. Resize the dried granulation through Fitzmill high speed # 93 screen knives forward.

### SR Granules II

1. Pour the Eudragit RS 30D into a separate vessel. Keep under continuous stirring to avoid settling down of solids.
2. Blend the dry mix scale up amounts of Phenylephrine HCl, Chlorpheniramine Maleate, Codeine Phosphate and Prosolv SMCC with a high shear mixer/lodige mixer for 10 minutes (main propeller on and chopper off).
3. Pump the granulating suspension (Eudragit RS 30D) into the high shear mixer/lodige mixer (main propeller on and chopper off).
4. After all suspension has been added mix for an additional minute (main propeller and chopper on).
5. Turn the equipment off and add the Methocel K4M Premium to the mixer/lodige mixer and mix for 1 minute (main propeller and chopper on).
6. Dry the granulation overnight at 45° C. until the LOD is 2% or less.
7. Resize the dried granulation through Fitzmill high speed # 93 screen knives forward.

### Final Blend

1. Load into the V-blender: IR Granules and SR Granules.
2. Blend for 10 minutes.
3. Pass through oscillating granulator screen # 30 the Magnesium Stearate.
4. Load into the V-blender the screened Magnesium Stearate and blend for 3 minutes.

### Final Subdivision

1. Fill into capsules size # 2.

### Example 6

A capsule in accordance with the present invention which contains (a) 75 mg Phenylephrine HCl, 60 mg Carbetapentane Citrate-SR and (b) 100 mg Diphenhydramine HCl-IR is illustrated as follows:

SR GRANULES

| | | |
|---|---|---|
| PHENYLEPHRINE HCl | 75.000 | 416.70 |
| CARBETAPENTANE CITRATE | 60.000 | 333.36 |
| MICROCRYSTALLINE CELLULOSE NF 50 | 110.450 | 613.66 |
| POVIDON K-30 | 11.200 | 62.23 |
| PURIFIED WATER | 40.000 | 222.24 |
| EUDRAGIT RL 30 (30%) | 27.500 | |
| HYPROMELLOSE K4M | 20.000 | |
| Totals | 284.900 | 1425.9 |

IR-GRANULES

| | | (AMOUNTS |
|---|---|---|
| | | FOR 1 Kg) |
| Ingredients | Dose(mg) | in g |
| DIPHENHYDRAMINE HCL | 100.000 | 555.60 |
| MICROCRYSTALLINE CELLULOSE NF 90 | 45.000 | 250.02 |
| PURIFIED WATER | 90.000 | 500.04 |
| POVIDONE K-30 USP | 5.800 | 32.22 |
| STEARIC ACID | 10.000 | 52.70 |
| MAGNESIUM STEARATE NF | 4.500 | 25.00 |
| Totals | 165.300 | 915.5 |
| Totals | 450.200 | 2341.5 |

Procedure

### SR Granules

1. Prepare a solution using the scale up amounts of Purified Water and Povidone K-30.
2. Pour into a separate container the Eudragit RL 30 D, keep under constant stirring while it is not in use.
3. Blend the dry mix scale up amounts of Phenylephrine HCl, Carbetapentane Citrate and Microcrystalline Cellulose NF 50 with a high shear mixer/granulator for 10 minutes (main propeller on, chopper off).
4. With the mixer/granulator on, pump the granulating solution prepared in step 1 into the mixer/granulator (main propeller on, chopper off). After completion, stop the mixer/granulator and rinse the container with Purified Water. Pump the rinse water to the mixer/granulator with main propeller and chopper on.
5. Pump the Eudragit RL 30 D into the high shear mixer/granulator; after pumping keep the mixer on for additional minute (keep the main propeller and chopper on)
6. Charge the post mix scale up amount of Hypromellose K4M Premium to the mixer/granulator and mix for 1 minute (main propeller and chopper on).
7. Dry the granulation overnight at 45° C. until the LOD is 2% or less.
8. Resize the dried granulation through Fitzmill high speed # 93 screen knives forward.

### IR Granules

1. Prepare a solution using the scale up amounts of Purified Water and Povidone K-30.
2. Blend the dry mix scale up amounts of Diphenhydramine HCl and Microcrystalline Cellulose NF 90 with a high shear mixer/granulator for 10 minutes.
3. With the mixer/granulator on, pump the granulating solution prepared in step 1 into the mixer/granulator (main propeller on and chopper off).
4. After all solution has been added mix for an additional minute (main propeller and chopper on).
5. Dry the granulation overnight at 40° C. until the LOD is 2% or less.
6. Resize the dried granulation through Fitzmill high speed # 12 screen knives forward.

### Final Blend

1. Load into the 20 ft³ V-blender: IR Granules, SR Granules.
2. Blend for 10 minutes.
3. Pass through oscillating granulator screen # 30 the Stearic Acid and Magnesium Stearate.
4. Load into the 20 ft³ V-blender the above screened Stearic Acid and Magnesium Stearate and blend for 3 minutes.

### Final Subdivision

1. Fill into capsules size # 1

### Example 7

A capsule in accordance with the present invention which contains (a) 10 mg Brompheniramine Maleate-SR and (b) 120 mg Pseudoephedrine HCl-SR is illustrated as follows:

SR GRANULES

| | | |
|---|---|---|
| MICROCRYSTALLINE CELLULOSE NF 50 | 25.000 | 138.90 |
| BROMPHENIRAMINE MALEATE | 10.000 | |
| POVIDON K-30 | 2.500 | 13.89 |
| PURIFIED WATER | 8.500 | 47.23 |
| EUDRAGIT RL 30 (30%) | 15.500 | |
| HYPROMELLOSE K4M | 12.000 | |
| Totals | 54.150 | 152.790 |

SR-GRANULES

| | | (AMOUNTS |
|---|---|---|
| | | FOR |
| Ingredients | Dose(mg) | 1 Kg) in g |
| PSEUDOEPHEDRINE HCL | 120.000 | 666.72 |
| MICROCRYSTALLINE CELLULOSE NF 90 | 143.250 | 795.90 |
| PURIFIED WATER | 85.000 | 472.26 |
| POVIDON K-30 | 19.000 | 105.56 |
| EUDRAGIT RL 30 (30%) | 100.000 | |
| HYPROMELLOSE K4M | 55.000 | |
| TOTAL | 367.250 | 1568.181 |
| STEARIC ACID | 18.000 | 52.70 |
| MAGNESIUM STEARATE NF | 8.000 | 44.45 |
| Totals | 393.250 | 3233.5 |
| Totals | 447.400 | 3386.3 |

Procedure

### SR Granules I

1. Prepare a solution using the scale up amounts of Purified Water, and Povidone K-30.
2. Add the scale up amounts of Brompheniramine Maleate by continuous stirring to the above prepared Povidon solution slowly (API Solution A).
3. Pour into a separate container the Eudragit RL 30 D; keep under constant stirring while it is not in use.
4. Blend the dry mix scale up amounts of Microcrystalline Cellulose NF 50 with a high shear mixer/granulator for 5 minutes (main propeller on, chopper off).
5. With the mixer/granulator on, pump the API solution A, a granulating solution prepared in step 1 and 2 into the mixer/granulator (main propeller on, chopper off). After completion, stop the mixer/granulator and rinse the container with Purified Water. Pump the rinse water to the mixer/granulator with main propeller and chopper on.
6. Pump the Eudragit RL 30 D into the high shear mixer/granulator; after pumping keep the mixer on for additional minute (keep the main propeller and chopper on)
7. Charge the post mix scale up amount of Hypromellose K4M Premium to the mixer/granulator and mix for 1 minute at fast speed (main propeller and chopper on).
8. Dry the granulation overnight at 45° C. until the LOD is 2% or less.
9. Resize the dried granulation through Fitzmill high speed # 93 screen 10. knives forward.

### SR Granules II

1. Prepare a solution using the scale up amounts of Purified Water and Povidone K-30.
2. Blend the dry mix scale up amounts of Pseudoephedrine HCl and Microcrystalline Cellulose NF 90 with a high shear mixer/granulator for 10 minutes.
3. With the mixer/granulator on, pump the granulating solution prepared in step 1 into the mixer/granulator (main propeller ON and chopper OFF).
4. After all solution has been added mix for an additional minute (main propeller and chopper on).
5. Dry the granulation overnight at 40° C. until the LOD is 2% or less.
6. Resize the dried granulation through Fitzmill high speed # 12 screen knives forward.

### Final Blend

1. Load into the 20 ft³ V-blender: SR Granules I, SR Granules II.
2. Blend for 10 minutes.
3. Pass through oscillating granulator screen # 30 the Stearic Acid and Magnesium Stearate.
4. Load into the 20 ft³ V-blender the above screened Stearic Acid and Magnesium Stearate and blend for 3 minutes.

### Final Subdivision

1. Fill into capsules size # 1.

### Example 8

A capsule in accordance with the present invention which contains (a) 25 mg Promethazine HCl-IR and (b) 120 mg Pseudoephedrine HCl, 30 mg Codeine Phosphate-SR is illustrated as follows:

| | | |
|---|---|---|
| | | (AMOUNTS |
| | | FOR |
| Ingredients | Dose(mg) | 1 Kg) in g |

SR GRANULES

| | | |
|---|---|---|
| PSEUDOEPHEDRINE HCL | 120.000 | 666.72 |
| CODEINE PHOSPHATE | 30.000 | 166.68 |
| MICROCRYSTALLINE CELLULOSE NF 50 | 123.500 | 686.17 |
| POVIDON K-30 | 35.000 | 194.46 |
| PURIFIED WATER | 150.000 | 833.40 |
| EUDRAGIT RL 30 (30%) | 105.000 | |
| HYPROMELLOSE K4M | 56.000 | |
| Totals | 396.000 | 1714.026 |

IR-GRANULES

| | | |
|---|---|---|
| PROMETHAZINE HCL | 25.000 | 138.90 |
| MICROCRYSTALLINE CELLULOSE NF 90 | 15.000 | 83.34 |
| PURIFIED WATER | 27.500 | 152.79 |
| POVIDONE K-30 USP | 1.800 | 10.00 |
| STEARIC ACID | 2.800 | 52.70 |
| MAGNESIUM STEARATE NF | 1.250 | 6.95 |
| Totals | 45.850 | 291.9 |
| Totals | 441.850 | 2005.912 |

Procedure

### SR Granules)

1. Prepare a solution using the scale up amounts of Purified Water, and Povidone K-30.
2. Add the scale up amounts of Pseudoephedrine HCl and Codeine Phosphate by continuous stirring to the above prepared Povidon solution slowly (API Solution A).
3. Pour into a separate container the Eudragit RL 30 D; keep under constant stirring while it is not in use.
4. Blend the dry mix scale up amounts of Microcrystalline Cellulose NF 50 with a high shear mixer/granulator for 3 minutes (main propeller on, chopper off).
5. With the mixer/granulator on, pump the API solution A, a granulating solution prepared in step 1 and 2 into the mixer/granulator (main propeller on, chopper off). After completion, stop the mixer/granulator and rinse the container with Purified Water. Pump the rinse water to the mixer/granulator with main propeller and chopper on.
6. Pump the Eudragit RL 30 D into the high shear mixer/granulator after pumping keep the mixer on for additional minute (keep the main propeller and chopper on).
7. Charge the post mix scale up amount of Hypromellose K4M Premium to the mixer/granulator and mix for 1 minute at fast speed (main propeller and chopper on).
8. Dry the granulation overnight at 45° C. until the LOD is 2% or less.
9. Resize the dried granulation through Fitzmill high speed # 93 screen knives forward.

### IR Granules

1. Prepare a solution using the scale up amounts of Purified Water and Povidone K-30.
2. Blend the dry mix scale up amounts of Promethazine HCl and Microcrystalline Cellulose NF 90 with a high shear mixer/granulator for 10 minutes.
3. With the mixer/granulator on, pump the granulating solution prepared in step 1 into the mixer/granulator (main propeller on and chopper off).
4. After all solution has been added mix for an additional minute (main propeller and chopper on).
5. Dry the granulation overnight at 40° C. until the LOD is 2% or less.
6. Resize the dried granulation through Fitzmill high speed # 12 screen knives forward.

### Final Blend

1. Load into the 20 ft³ V-blender SR Granules, IR Granules.
2. Blend for 10 minutes.
3. Pass through oscillating granulator screen # 30 the Stearic Acid and Magnesium Stearate.
4. Load into the 20 ft³ V-blender the above screened Stearic Acid and Magnesium Stearate and blend for 3 minutes.

### Final Subdivision

1. Fill into capsules size # 0.

### Example 9

A capsule in accordance with the present invention which contains a total of 75 mg Indomethacine in two different sustained release matrices is illustrated as follows:

| | | |
|---|---|---|
| | | (AMOUNTS |
| | | FOR |
| Ingredients | Dose(mg) | 1 Kg) in g |

SR GRANULES (PART: I)

| | | |
|---|---|---|
| INDOMETHACINE | 25.000 | 138.90 |
| CONFECTIONER'S SUGAR | 28.000 | 155.57 |
| MICROCRYSTALLINE CELLULOSE NF90 | 90.500 | 502.82 |
| PREGELATINIZED STARCH | 7.300 | |
| Totals | 150.800 | 797.286 |
| EUDRAGIT RL 30 (30%) | 65.000 | |
| HYPROMELLOSE K4M | 20.000 | |
| Totals | 190.300 | 797.286 |

SR-GRANULES (PART II)

| | | |
|---|---|---|
| INDOMETHACINE | 50.000 | 277.80 |
| MICROCRYSTALLINE CELLULOSE NF 90 | 160.000 | 888.96 |
| PREGELATINIZED STARCH | 14.600 | 81.12 |
| CONFECTIONER'S SUGAR | 56.000 | 972.300 |
| Totals | 280.600 | 2220.178 |
| EUDRAGIT RL 30 (30%) | 110.000 | 611.16 |
| HYPROMELLOSE K4M | 35.000 | 194.46 |
| Totals | 348.600 | 3998.098 |

Procedure

### SR Granules Part I

1. Pour into a separate container the Eudragit RL 30 D; keep under constant stirring while it is not in use.
2. Blend the dry mix the scale up amounts of Indomethacine, Microcrystalline Cellulose NF90, Pregelatinized Starch and Confectioner's Sugar by continuous stirring in the high shear mixer/granulator for 10 minutes (main propeller on, chopper off).
3. With the mixer/granulator on, pump the Eudragit RL 30 D granulating solution prepared in step 1 into the mixer/granulator (main propeller on, chopper off). After completion, stop the mixer/granulator and rinse the container with Purified Water. Pump the rinse water to the mixer/granulator with main propeller and chopper on.
4. Charge the post mix scale up amount of Hypromellose K4M Premium to the mixer/granulator and mix for 1 minute at fast speed (main propeller and chopper on).
5. Dry the granulation overnight at 45° C. until the LOD is 2% or less.
6. Resize the dried granulation through Fitzmill high speed # 93 screen knives forward.

### SR Granules Part II

1. Pour into a separate container the Eudragit RL 30 D; keep under constant stirring while it is not in use.
2. Blend the dry mix scale up amounts of P Indomethacine, Microcrystalline Cellulose NF90, Pregelatinized Starch and Confectioner's Sugar by continuous stirring in the high shear mixer/granulator for 10 minutes (main propeller on, chopper off).
3. With the mixer/granulator on, pump the granulating solution prepared in step 1 into the mixer/granulator (main propeller ON and chopper OFF).
4. After all solution has been added mix for an additional minute (main propeller and chopper on).
5. Dry the granulation overnight at 40° C. until the LOD is 2% or less.
6. Re-size the dried granulation through Fitzmill high speed # 12 screen knives forward.

### Final Blend

1. Load into the 20 ft³ V-blender SR Granules Part I, SR Granules Part II.
2. Blend for 10 minutes.
3. Dosage of the 75 mg per capsules can be adjusted by getting the assay results of the Granulation Part I and Part II and adjusting with the remaining quantity of the weight with the Part III placebo Granules
4. Pass through oscillating granulator screen # 30 the Stearic Acid and Magnesium Stearate.
5. Load into the 20 ft³ V-blender the above screened Stearic Acid and Magnesium Stearate and blend for 3 minutes.

### Final Subdivision

1. Fill into capsules size # 0.

### Example 10

A capsule in accordance with the present invention which contains (a) 20 mg Phenylephrine HCl, 6 mg Chlorpheniramine Maleate-SR and (b) 1 mg Methscopolamine Nitrate-IR is illustrated as follows:

| | | |
|---|---|---|
| Ingredients | Dose(mg) | FOR 1 Kg) |

SR GRANULES(PART I)

| | | |
|---|---|---|
| MICROCRYSTALLINE CELLULOSE NF 90 | 80.000 | 444.48 |
| PHENYEPHRINE HCL | 20.000 | 111.12 |
| CHLORPHENIRAMINE MALEATE | 6.000 | 33.34 |
| CALCIUM PHOSPHATE DIBASIC | 4.000 | 22.22 |
| POVIDON K-30 | 5.000 | 27.78 |
| PURIFIED WATER | 85.000 | 472.26 |
| EUDRAGIT RL 30 (30%) | 80.500 | 447.26 |
| HYPROMELLOSE K4M | 18.000 | 100.01 |
| Totals | 157.150 | 873.125 |

IR-GRANULES(PART II)

| | | |
|---|---|---|
| MICROCRYSTALLINE CELLULOSE NF 90 | 35.000 | 194.46 |
| CALCIUM PHOSPHATE DIBASIC | 3.000 | 16.67 |
| Totals | 195.150 | 1084.25 |
| METHSCOPOLAMINE NITRATE | 1.000 | 5.56 |
| PURIFIED WATER | 45.000 | 250.02 |
| POVIDON K-30 | 5.000 | 27.78 |
| TOTAL | 6.000 | 33.34 |
| TOTAL | 201.150 | |

LUBICATION

| | | |
|---|---|---|
| STEARIC ACID | 18.000 | 52.70 |
| MAGNESIUM STEARATE NF | 8.000 | 44.45 |
| Totals | 26.000 | 1453.6 |
| Totals | 227.150 | 2326.8 |

Procedure

### SR Granules Part I

1. Prepare a solution using the scale up amounts of Purified Water, and Povidone K-30.
2. Add the scale up amounts of Phenylephrine HCl, Chlorpheniramine Maleate by continuous stirring to the above prepared Povidon solution slowly (API Solution A).
3. Pour into a separate container the Eudragit RL 30 D; keep under constant stirring while it is not in use.
4. Blend the dry mix scale up amounts of Microcrystalline Cellulose NF 90 with Calcium Phosphate Dibasic in a high shear mixer/granulator for 5 minutes (main propeller on, chopper off).
5. With the mixer/granulator on, pump the API solution A, a granulating solution prepared in step 1 and 2 into the mixer/granulator (main propeller on, chopper off). After completion, stop the mixer/granulator and rinse the container with Purified Water. Pump the rinse water to the mixer/granulator with main propeller and chopper on.
6. Pump the Eudragit RL 30 D from step 3 into the high shear mixer/granulator after pumping keep the mixer on for additional minute (keep the main propeller and chopper on).
7. Charge the post mix scale up amount of Hypromellose K4M Premium to the mixer/granulator and mix for 1 minute at fast speed (main propeller and chopper on).
8. Dry the granulation overnight at 45° C. until the LOD is 2% or less.
9. Resize the dried granulation through Fitzmill high speed # 93 screen knives forward.

### IR Granules Part II

1. Prepare a solution using the scale up amounts of Purified Water and Povidone K-30.
2. Add the scale up amounts of Methscopolamine Nitrate by continuous stirring to the above prepared Povidon solution slowly (API Solution B).
3. Blend the dry mix scale up amounts of Microcrystalline Cellulose NF 90 and Calcium Phosphate Dibasic with a high shear mixer/granulator for 10 minutes.
4. With the mixer/granulator on, pump the granulating solution prepared in step 1 and 2 into the mixer/granulator (main propeller on and chopper off).
5. After all solution has been added mix for an additional minute (main propeller and chopper on).
6. Dry the granulation overnight at 40° C. until the LOD is 2% or less.
7. Resize the dried granulation through Fitzmill high speed # 12 screen knives forward.

### Final Blend

1. Load into the 20 ft³ V-blender SR Granules Part I, IR Granules Part II.
2. Blend for 10 minutes.
3. Pass through oscillating granulator screen # 30 the Stearic Acid and Magnesium Stearate.
4. Load into the 20 ft³ V-blender the above screened Stearic Acid and Magnesium Stearate and blend for 3 minutes.

### Final Subdivision

1. Fill into capsules size # 2.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A capsule for oral administration to a human subject, wherein the capsule comprises at least two different pharmaceutical compositions in granular form.

2. The capsule of claim 1, wherein at least one of the at least two different compositions is an immediate release composition.

3. The capsule of claim 1, wherein at least one of the at least two different compositions is a controlled release composition.

4. The capsule of claim 1, wherein the capsule comprises at least two compositions which differ with respect to at least the release profiles thereof.

5. The capsule of claim 4, wherein the two different pharmaceutical compositions comprise the same active ingredient and provide two different release profiles of the active ingredient.

6. The capsule of claim 5, wherein one of the compositions is an immediate release composition and the other one is a controlled release composition.

7. The capsule of claim 1, wherein the at least two different pharmaceutical compositions comprise at least two different active ingredients.

8. The capsule of claim 7, wherein at least one of the at least two different active ingredients is present in only one of the compositions.

9. The capsule of claim 8, wherein one of the compositions is an immediate release composition and the other one is a controlled release composition.

10. The capsule of claim 1, wherein each of the compositions comprises not more than about 40% by weight of one or more active ingredients, based on a total weight of a composition.

11. The capsule of claim 2, wherein the immediate release composition comprises at least about 70% by weight of one or more excipients selected from sugar and sugar alcohols, microcrystalline cellulose, dicalcium phosphate, starch and derivatives thereof, stearic acid and salts thereof, and optionally crosslinked polyvinylpyrrolidone, based on a total weight of all excipients of the composition.

12. The capsule of claim 11, wherein the immediate release composition comprises at least about 80% by weight of the one or more excipients.

13. The capsule of claim 11, wherein the immediate release composition comprises at least about 40% by weight of microcrystalline cellulose, based on a total weight of all excipients of the composition.

14. The capsule of claim 3, wherein the controlled release composition comprises at least about 70% by weight of one or more excipients selected from microcrystalline cellulose, starch and derivatives thereof, cellulose ethers, cellulose esters, copolymers of esters of at least one of acrylic acid and methacrylic acid, sugars and sugar alcohols, alginates, gelatin, polyvinylalcohol, polyvinylpyrrolidone, and stearic acid and salts thereof, based on a total weight of all excipients of the composition.

15. The capsule of claim 14, wherein the controlled release composition comprises at least about 80% by weight of the one or more excipients.

16. The capsule of claim 15, wherein the controlled release composition comprises at least about 40% by weight of microcrystalline cellulose, based on a total weight of all excipients of the composition.

17. The capsule of claim 1, wherein the capsule comprises a total of not more than about 0.8 g of the at least two different pharmaceutical compositions.

18. The capsule of claim 4, wherein the capsule comprises a total of not more than about 0.6 g of the two different pharmaceutical compositions.

19. The capsule of claim 1, wherein at least one of the at least two different pharmaceutical compositions comprises one or more active ingredients selected from analgesics, antihistamines, decongestants, antitussives, expectorants, vitamins, anti-inflammatories, anti-infectives, antibiotics and laxatives.

20. The capsule of claim 1, wherein at least one of the at least two different pharmaceutical compositions has been obtained by a wet granulation method.

21. The capsule of claim 1, wherein at least one of the at least two different pharmaceutical compositions has been obtained by a dry granulation method.

22. The capsule of claim 1, wherein the capsule comprises a soft gelatin capsule.

23. The capsule of claim 1, wherein the capsule comprises a hard gelatin capsule.

24. The capsule of claim 1, wherein the capsule comprises a therapeutically effective amount of each active ingredient contained therein.

25. A gelatine capsule for oral administration to a human subject, wherein the capsule comprises at least two different pharmaceutical compositions which have been obtained by granulation of a mixture of one or more active ingredients and one or more excipients, at least one of the compositions being an immediate release composition and comprising at least one first active ingredient and at least one composition being a controlled release composition and comprising at least one second active ingredient which is the same as or different from the first active ingredient, and wherein the capsule comprises a therapeutically effective amount of the or each active ingredient contained therein.

26. The capsule of claim 25, wherein each of the compositions comprises not more than about 40% by weight of one or more active ingredients, based on a total weight of the composition.

27. The capsule of claim 26, wherein the immediate release composition comprises at least about 70% by weight of one or more excipients selected from sugar and sugar alcohols, microcrystalline cellulose, dicalcium phosphate, starch and derivatives thereof, stearic acid and salts thereof, and optionally crosslinked polyvinylpyrrolidone, based on a total weight of all excipients of the immediate release composition.

28. The capsule of claim 27, wherein the immediate release composition comprises at least about 40% by weight of microcrystalline cellulose, based on a total weight of all excipients of the composition.

29. The capsule of claim 27, wherein the controlled release composition comprises at least about 70% by weight of one or more excipients selected from microcrystalline cellulose, starch and derivatives thereof, cellulose ethers, cellulose esters, copolymers of esters of at least one of acrylic acid and methacrylic acid, sugars and sugar alcohols, alginates, gelatin, polyvinylalcohol, polyvinylpyrrolidone, and stearic acid and salts thereof, based on a total weight of all excipients of the controlled release composition.

30. The capsule of claim 29, wherein the controlled release composition comprises at least about 40% by weight of microcrystalline cellulose, based on a total weight of all excipients of the composition.

31. The capsule of claim 29, wherein the capsule comprises a total of not more than about 0.6 g of the at least two pharmaceutical compositions.

32. The capsule of claim 31, wherein at least one of the at least two different pharmaceutical compositions comprises one or more active ingredients selected from analgesics, antihistamines, decongestants, antitussives, expectorants, vitamins, anti-inflammatories, anti-infectives, antibiotics and laxatives.

33. A process for making a capsule for administration to a human subject, which process comprises filling the capsule with at least two different pharmaceutical compositions, wherein the at least two different compositions have been provided by a granulation method.

34. The process of claim 33, wherein at least one of the compositions has been obtained by a wet granulation method.

35. The process of claim 33, wherein at least one of the compositions has been obtained by a dry granulation method.

36. The process of claim 33, wherein at least one of the compositions comprises an immediate release composition.

37. The process of claim 33, wherein at least one of the compositions comprises a controlled release composition.

38. The process of claim 33, wherein the at least two different pharmaceutical compositions comprise at least two different active ingredients.

39. The process of claim 38, wherein each of the compositions comprises not more than about 40% by weight of one or more active ingredients, based on a total weight of a composition.

40. The process of claim 36, wherein the immediate release composition comprises at least about 70% by weight of one or more excipients selected from sugars and sugar alcohols, microcrystalline cellulose, dicalcium phosphate, starch, stearic acid and salts thereof, and optionally crosslinked polyvinylpyrrolidone, based on a total weight of all excipients of the composition.

41. The process of claim 40, wherein the immediate release composition comprises at least about 40% by weight of microcrystalline cellulose, based on a total weight of all excipients of the composition.

42. The process of claim 37, wherein the controlled release composition comprises at least about 70% by weight of one or more excipients selected from microcrystalline cellulose, starch and derivatives thereof, cellulose ethers, cellulose esters, copolymers of esters of at least one of acrylic acid and methacrylic acid, sugars and sugar alcohols, alginates, gelatin, polyvinylalcohol, polyvinylpyrrolidone, and stearic acid and salts thereof, based on a total weight of all excipients of the composition.

43. The process of claim 42, wherein the controlled release composition comprises at least about 40% by weight of microcrystalline cellulose, based on a total weight of all excipients of the composition.

44. The process of claim 33, wherein the capsule is filled with a total of not more than about 0.8 g of the at least two different pharmaceutical compositions.
